(19) United States
(12) Patent Application Publication
(10) Pub. No.: US 2025/0132044 A1
(43) Pub. Date: Apr. 24, 2025

(54) SYSTEM AND METHOD FOR THE HEALTH MANAGEMENT WITHIN THE FIRST 1000 DAYS OF LIFE

(71) Applicant: NEOPREDIX AG, Basel (CH)

(72) Inventors: Marc PFISTER, Hilterfingen (CH); Sven WELLMANN, Obertraubling (DE); Gilbert KOCH, Überlingen (DE)

(21) Appl. No.: 18/570,229

(22) PCT Filed: Jun. 14, 2022
(86) PCT No.: PCT/EP2022/066202

§ 371 Date: Dec. 14, 2023

(30) Foreign Application Priority Data
Jun. 15, 2021 (EP) 21179638.8

(51) Int. Cl. G16H 50/30; G16H 10/60; G16H 50/50

(52) U.S. Cl. CPC G16H 50/30; G16H 10/60; G16H 50/50

(57) ABSTRACT

The present invention a system and a method for monitoring young children comprising a plurality of sensors (1-5) providing signals that are configured to be converted into sensor data and a management component (10-26) configured for receiving the sensor data and for managing a health status and or disease progression on the basis of a model. The management component can comprise a prediction component for predicting the health status on the basis of a model. The method comprises a respective prediction step. The management component can be further configured to compute at least one of health data from a health data base, individual data from a storage and/or genetic data from a genetic data base. The plurality of sensors can be configured to be attached or applied to a young child and/or its mother. The model can be a trained model that can be further trained and adjusted on the basis of the data gained.

### FIELD

The present invention relates to a system and a method for the health management of fetus, newborn, and infant within first 1,000 days of life, encompassing time from conception until around the second birthday. The invention can be particularly suited for an early recognition and prevention of negative developments before and after birth of children, such as any negative or severe developments based on risk factors, covariates, predispositions, diseases etc.

### INTRODUCTION

In the past, medicine was diagnosing any health-related impacts on the basis of the status quo of a patient. Modern medicine is more and more trying to prevent any negative impacts onto health, based on environmental factors, genetic and epigenetic predisposition, body development and maturation, resilience factors and lifestyle choices. Negative impacts onto health are dynamical processes, which begin before individuals realize they are affected. Individual and public health promotion as well as disease prevention relies on anticipatory actions that can be categorized as primary, secondary, tertiary and quaternary prevention. The latter is the set of health activities to mitigate or avoid the consequences of overdiagnosis and overmedication, unnecessary or excessive intervention. However, this holistic approach is very complex as there are many factors, some of them even unknown or interactions of certain factors not known, as such and/or depending on the individual and environment.

Young children are particularly vulnerable to severe health-related impacts as any early diagnosis is difficult and their bodies and their immune systems are immature and are less able to compensate any unfavorable situations or developments. It is thus of utmost importance to monitor them and to attempt to prevent any unwanted developments as early and reliable as possible.

The first 1,000 days of life—the time spanning roughly between conception and one's second birthday—are a time of tremendous potential and enormous vulnerability. How well or how poorly mothers and children are nourished and cared for during this time has a profound impact on a child's ability to grow, learn and thrive. This is because the child's organism starts to grow from embryonic stage based on the genetic and epigenetic makeup and the environment provided in-utero and after birth. Thus, life conditions and experiences during the first 1,000 days of life strongly influence mental and physical health later in life. Research in the fields of neuroscience, biology and early childhood development provide powerful insights into how nutrition, relationships, and environments in the 1,000 days between a woman's pregnancy and a child's 2nd birthday shape future outcomes. In particular, nutrition plays a foundational role in a child's development and her country's ability to prosper. Poor nutrition in the first 1,000 days can cause irreversible damage to a child's growing brain, affecting its ability to do well in school and earn a good living—and making it harder for a child and her family to rise out of poverty. It can also set the stage for later physical and mental diseases which can lead to a lifetime of health problems.

U.S. Pat. No. 10,490,049 B2 describes a method that includes receiving longitudinal data for a time period, wherein patient data are measured at one, two or more timepoints from a patient. Further, the method includes identifying a plurality of segmented trends in the first patient data and the second patient data as one of an uptrend, a downtrend, and neutral. Furthermore, the method includes classifying at least one segmented trend from the plurality of segmented trends as a pattern. Additionally, the method includes triggering an alarm as an early warning of patient distress or disease progression based on the pattern.

WO 2017/077414 is directed to a process and system for determining a minimal, ‘pruned’ version of the known ARDS model is provided that quantifies the risk of ARDS in terms of physiologic response of the patient, eliminating the more subjective and/or therapeutic features currently used by the conventional ARDS models. This approach provides an accurate tracking of ARDS risk modeled only on the patient's physiological response and observable reactions, and the decision criteria are selected to provide a positive prediction as soon as possible before an onset of ARDS. In addition, the pruning process also allows the ARDS model to be customized for different medical facility sites using selective combinations of risk factors and rules that yield optimized performance. Additionally, predictions may be provided in cases with missing or outdated data by providing estimates of the missing data, and confidence bounds about the predictions based on the variance of the estimates.

US20180333106 A1 relates to methods and systems for predicting deterioration of a patient's condition within a future time interval based on a time series of values for monitored physiological variables measured from a patient, and in some instances, providing advanced notice to clinicians or caregivers when deterioration is forecasted or modifying treatment for the patient are provided. In particular, deterioration of a patient's condition is based on a Hopf bifurcation model and is predicted using a ratio of deviations for monitored physiological variables. A ratio of deviations relates the standard deviation and root mean square of successive differences for a set of physiological values measured over time. The RoD for one or more variables, such as heart rate, respiratory rate, and blood pressure, may be used to predict the likelihood of the patient's condition deteriorating into an unstable state as what occurs in a Hopf bifurcation.

WO 2019/063722 relates to a method and a computer program for estimating a bilirubin level of a young child, comprising the steps of: acquiring a series of bilirubin levels estimated at different time points from a sample obtained from a young child, acquiring a plurality of covariates from the young child, each comprising an information about a neonatal property, providing a pre-defined bilirubin model function, wherein the bilirubin model function is configured to describe a time course of a bilirubin level of a young child, determining a plurality of model parameters of the bilirubin model function, wherein each model parameter is estimated from at least one covariate of the plurality of covariates and an associated population model parameter, determining from the series of acquired bilirubin levels and the bilirubin model function with the determined model parameters an expected bilirubin level of the young child for a time particularly later than a lastly acquired bilirubin level of the series of bilirubin levels.

WO 2021/1030637 concerns a patient health management platform accesses a metabolic profile for a patient and bio signals recorded for the patient during a current time period comprising sensor data and/or lab test data collected for the patient. The platform receives patient data recorded during the current time period comprising food items consumed, medications taken, and symptoms experienced by the patient. The platform implements a machine-learned metabolic model to determine a metabolic state of the patient at a conclusion of the current time period by comparing a true representation of the metabolic state and a prediction of the metabolic state. The true representation and the prediction are determined based on the recorded bio signals and the recorded patient data, respectively. The platform generates a patient-specific treatment recommendation outlining instructions for the patient to improve their metabolic state and provides the patient-specific treatment recommendation to the patient device for display to the patient.

US20210059597 A1 is directed to a sepsis monitoring system determines an initial sepsis risk assessment score, and automatically and continuously updates the sepsis risk assessment score using a sepsis risk assessment model that receives biomarker and vital signs data, electronic medical records data, and admissions data to continuously update the sepsis risk assessment score. The system determines whether the patient is likely to develop sepsis based on the updated sepsis risk assessment score, and in response to determining that the patient is likely to develop sepsis, generates a notification to drive an early intervention by one or more caregivers.

However, all systems in the art are still not matured and not suited for a reliable young child monitoring and risk prevention.

### SUMMARY

In light of the above, it is an object of the present invention to overcome or at least alleviate the shortcomings of the prior art.

The system as well as the method can also be provided or performed without presence of any patient, i.e., a young child and/or its mother, and can be operated for setup reasons, tests or remote and/or time-lagged monitoring.

In view of the lack of a specific term, in the following the term “young child” or “young children” stand for the first 1,000 days from conception until around the second birthday of a child. This comprises fetus, preterm and term neonates, newborns, babies, infants, toddlers, children until around their second birthday.

In the present application a sensor comprises a device, module, machine, or subsystem whose purpose is to detect events or changes inwardly or its environment and send the information or data to other entities, such as computers. There is a wide range of sensors, measuring directly and/or indirectly and/or deriving any chemical and/or physical properties.

Computational modelling is the use of computers to predict and study complex systems combining mathematics, statistics, pharmacometrics, artificial neural network, and supervised machine learning with developmental pharmacology, (patho) physiology, and computer science. A model is intended to mean a computational model that contains a plurality of variables that characterize the system being studied. It can be based onto a machine learning (ML) or artificial intelligence (AI) approach.

A management of a health status comprises the prediction, detection, early recognition and prediction of a health status and any deviation therefrom and prevention of negative developments.

Prediction is done by adjusting the variables alone or in combination and observing the outcomes. The variables can be data, such as sensor data, indirect, direct and/or derived sensor data, variates, covariates, etc.

A recognition is intended to comprise any direct and/or indirect detection and/or derivation of a health status. The latter comprises the status of a healthy young child and any deviation therefrom.

The covariates are particularly a physical property of the young child, such as the birth weight, or events that are associated to the young child, such as receiving a phototherapy or being born via caesarean section. Therefore, a covariate in the context of the description is not arbitrarily chosen but is a property associated to the young child. The acquisition of a covariate can for example be facilitated by a database query of the birth record and/or by an interview with a person in possession of this information.

The present invention relates to a system for monitoring young children and/or their mothers. The method according to the present invention corresponds to the system and comprises steps in the form of method steps. Wherever not explicitly mentioned, the present disclosure also discloses method steps that correspond to the function of system or device features. Thus, wherever is mentioned a system comprising a component for performing X also a method with the step of performing X is disclosed.

Anyhow, the present invention comprises a plurality of sensors configured for providing signals that are configured to be converted into sensor data. Further a management component is comprised that is configured for receiving the sensor data and for managing a health status on the basis of a model.

Thus, management or a management component in this context means a technical step or feature, respectively, that automatically processes the health status.

In this context, the present invention also comprises a method for monitoring young children comprising the steps of providing signals and/or converted sensor data and managing a health status by a management component configured to receive the sensor data on the basis of a model.

The management component can comprise a prediction component for predicting the health status on the basis of a model. The method comprises a respective prediction step.

The management component can be further configured to compute at least one of health data from a health data base, individual data from a storage and/or genetic data from a genetic data base. The latter term genetic comprises also any other genetic, genomic, epigenetic data etc.

The plurality of sensors can be configured to be attached or applied to a young child and/or its mother.

The computing component can be configured to receive sensor data. The management component can further comprise a sensor data storage that is configured for storing the sensor data from the sensors, preferably over time. It can collect data of one or more young children or can contain historical data.

The management component can further comprise a processing component that is configured for processing sensor data received from the sensors. The processing can comprise any handling, compressing, encoding, directing etc. of data.

The management component can further comprise a sensor data storage that is configured for storing the sensor data from the sensors. It can also contain other data. The storage can also comprise different storages located at different locations, such as locally and/or remotely.

The management component can further comprise a computing component that is configured for computing the sensor data and for applying the model. The invention also covers the respective method step.

The management component can further comprise a computing component that is configured for receiving and computing the sensor data and for applying and/or establishing and/or adjusting the model.

The management component can further comprise a deciding component that is configured for deciding based on the result of the computing component. The decision can be distinct and/or fuzzy.

The management component can also comprise a deciding component that is configured for initiating the managing component for managing the health status with a different complexity level. The complexity level can comprise the complexity of the model and/or the precision of the computing. Less complexity may mean a model with fewer parameters or dimensions than more complexity.

The management component can further comprise a deciding component that is configured for initiating the managing component for managing the health status with a different frequency. The frequency can comprise the frequency of activating the sensors and collecting their data and/or of applying the model. The complexity level and the frequency can be modified in combination as well.

The management component can further comprise a deciding component that is configured for initiating the managing component for triggering and/or controlling at least one medical device. This can establish a feedback-loop controlling of the medical device or medical devices.

The computing component and/or management component can be located locally and/or remotely.

The management component can be configured for automatically providing a diagnosis and/or a therapy. It can be also configured for logging and/or documenting the health status.

The management component can instead or in addition be configured for predicting the health status. The model can be trained by later comparing the previously predicted and the actual health status.

The management component can be further configured to trigger an intervention on the basis of the prediction. The intervention can comprise an acoustical signal, such as an alarm signal. It can also trigger an optical signal or a software flag.

The intervention can further comprise a treatment proposal, preferably configured to be displayed on a screen.

The intervention can also comprise a control signal to a respective treatment component.

The model can be configured to predict the health status of a young child on the basis of the sensor data.

The management component can be configured to train the model, preferably on the basis of the sensor data and optionally other variables.

The model can be based on a Bayesian statistical approach, an artificial neural network (ANN) approach, on a pharmacometrics approach, on a supervised learning approach and/or on a deep learning and/or multi-layer neural network approach, and any combination thereof.

The system and method according to the present invention and their model is based on an explainable AI concept (XAI). This can be an optional advantage in order to better understand any findings and make use of them as well as to minimize risks. Explainable artificial intelligence (XAI) is a set of processes and methods that allows human users to comprehend and trust the results and output created by machine learning algorithms. Explainable AI is used to describe an AI model, its expected impact and potential biases. It helps characterize model accuracy, fairness, transparency and outcomes in AI-powered decision making. Explainability can help developers ensure that the system is working as expected, it might be necessary to meet regulatory standards, or it might be important in allowing those affected by a decision to challenge or change that outcome.

A trained model can be configured to predict the health status of a young child on the basis of the sensor data.

The sensors can be configured to transmit the signals and/or the sensor data wireless and/or hard-wired.

The sensors can comprise at least one of a non-contact sensor; and/or a contact sensor; a camera for capturing frames and/or videos; a camera that is configured to detect physiological and/or skin properties and/or movement of a young child and/or its mother; an amniotic fluid sensor and/or analyzer; a mother milk sensor and/or analyzer; laboratory sensors; marker sensors; an infrared sensor; a photometric sensor; a respiratory sensor; a breathing analyzer; a capnography sensor; a cardiac sensor; a blood pressure sensor; a hemodynamic sensor; a movement sensor; a temperature sensor; a blood oxygen sensor; a pulse oximetric sensor; a neurological sensor; an EEG sensor; an electrolyte sensor; a hydrolyte sensor; a peptide sensor; a urine analyzer; a stool analyzer; a blood gas analyzer; a bladder pressure sensor; an intracranial pressure sensor; a transcutaneous sensor; a tissue oxidization sensor and/or an ultrasonic sensor, and any combination thereof.

The sensors or a part thereof can be arranged at a point of care (POC) location. This is usually a hospital or any other treatment site.

The system can also comprise a sensor data storage that is configured to store the sensor signals and/or sensor data. The sensor data storage can be located locally with respect to the sensors. A sensor data processing component can be configured to process the sensor signals into sensor data.

The data processing component can be configured to initiate storage of the sensor data in the sensor data storage.

The computing component can be configured to communicate with the processing component.

The computing component can be also configured to compute the sensor data of a plurality of processing components. It can also be configured to train a software component on the basis of the sensor data of a plurality of processing components. Alternatively, or additionally, it can be configured to train a software component on the basis of the sensor data of a plurality of processing components wherein the computing component is located remotely, such as in the cloud.

The system can also comprise a remote storage that is located at least remotely with respect to one set of sensors. It can also comprise a triggering component that is configured to deliver triggering data on the basis of the model. It can trigger an intervention. The intervention can be initiated or triggered locally with respect to the sensors.

The invention is further described with the following numbered embodiments.

Below, system embodiments will be discussed. These embodiments are abbreviated by the letter “S” followed by a number. Whenever reference is herein made to “system embodiments”, these embodiments are meant.
S1. System for monitoring young children comprising:
   a plurality of sensors (1-5) providing signals that are configured to be converted into sensor data;
   a management component (10-26) configured for receiving the sensor data and for managing a health status and or disease progression on the basis of a model.
S2. System according to the preceding system embodiment for monitoring young children between their conception and the subsequent 1,000 days.
S3. System according to any of the preceding system embodiments wherein the management component (10-26) comprises a prediction component for predicting the health status on the basis of a model.
S4. System according to any of the preceding system embodiments wherein the management component (10-26) is further configured to compute at least one of the following data:
   health data from a health data base (21);
   individual data from a storage (22); and/or
   genetic data from a genetic data base (23).
S5. System according to any of the preceding system embodiments wherein the plurality of sensors (1-5) is configured to be attached to a young child and/or its mother.
S6. System according to any of the preceding system embodiments wherein the computing component (20) is configured to receive sensor data.
S7. System according to any of the preceding system embodiments wherein the management component (10-26) further comprises a sensor data storage (11) that is configured for storing the sensor data from the sensors (1-5).
S8. System according to any of the preceding system embodiments wherein the management component (10-26) further comprises a processing component (10) that is configured for processing sensor data received from the sensors (1-5).
S9. System according to any of the preceding system embodiments wherein the management component (10-26) further comprises a sensor data storage (11) that is configured for storing the sensor data from the sensors (1-5).
S10. System according to any of the preceding system embodiments wherein the management component (10-26) further comprises a computing component (20) that is configured for computing the sensor data and for applying the model.
S11. System according to any of the preceding system embodiments wherein the management component (10-26) further comprises a computing component (20) that is configured for receiving and computing the sensor data and for applying the model.
S12. System according to any of the preceding system embodiments wherein the management component (10-26) further comprises a computing component (20) that is configured for computing the sensor data and for establishing the model.
S13. System according to any of the preceding system embodiments wherein the management component (10-26) further comprises a computing component (20) that is configured for receiving and computing the sensor data and for establishing the model.
S14. System according to any of the preceding system embodiments wherein the management component (10-26) further comprises a computing component (20) that is configured for computing the sensor data and for adjusting the model.
S15. System according to any of the preceding system embodiments wherein the management component (10-26) further comprises a computing component (20) that is configured for receiving and computing the sensor data and for adjusting the model.
S16. System according to any of the preceding system embodiments wherein the management component (10-26) further comprises a deciding component (20) that is configured for deciding based on the result of the computing component (20).
S17. System according to any of the preceding system embodiments wherein the management component (10-26) further comprises a deciding component (20) that is configured for initiating the managing component (10-26) for managing the health status with a different complexity level.
S18. System according to any of the preceding system embodiments wherein the management component (10-26) further comprises a deciding component (20) that is configured for initiating the managing component (10-26) for managing the health status with a different frequency.
S19. System according to any of the preceding system embodiments wherein the management component (10-26) further comprises a deciding component (20) that is configured for initiating the managing component (10-26) for triggering at least one medical device.
S20. System according to any of the preceding system embodiments wherein the management component (10-26) further comprises a deciding component (20) that is configured for initiating the managing component (10-26) for controlling at least one medical device.
S21. System according to any of the preceding system embodiments wherein the computing component (20) is located locally and/or remotely.
S22. System according to any of the preceding system embodiments wherein the management component (10-26) is located locally and/or remotely.
S23. System according to any of the preceding system embodiments wherein the management component (10-26) is configured for providing a diagnosis.
S24. System according to any of the preceding system embodiments wherein the management component (10-26) is configured for providing a therapy.
S25. System according to any of the preceding system embodiments wherein the management component (10-26) is configured for logging the health status.
S26. System according to any of the preceding system embodiments wherein the management component (10-26) is configured for predicting the health status.
S27. System according to any of the preceding system embodiments wherein the management component (10-26) is configured for predicting the health status and for training the model on the basis of the previously predicted and the actual health status.
S28. System according to any of the preceding system embodiments wherein the management component (10-26) is further configured to trigger an intervention on the basis of the prediction.
S29. System according to the preceding system embodiment wherein the intervention comprises an alarm signal.
S30. System according to the two preceding system embodiments wherein the intervention comprises an acoustical and/or optical signal.
S31. System according to the two preceding system embodiments wherein the intervention comprises an acoustical and/or optical signal.
S32. System according to the three preceding system embodiments wherein the intervention comprises a treatment proposal, preferably configured to be displayed on a screen (22).
S33. System according to the four preceding system embodiments wherein the intervention comprises a control signal to a respective treatment component (22).
S34. System according to any of the preceding system embodiments further comprising a model that is configured to predict health status and or disease progression of a young child on the basis of the sensor data.
S35. System according to any of the preceding system embodiments wherein the management component (10-26) is configured to train the model.
S36. System according to any of the preceding system embodiments wherein the management component (10-26) is configured to train the model on the basis of the sensor data.
S37. System according to any of the preceding system embodiments wherein the management component (10-26) is configured to train the model on the basis of the sensor data and other variables.
S38. System according to any of the preceding system embodiments wherein the model is based on a Bayesian statistical approach.
S39. System according to any of the preceding system embodiments wherein the model is based on an artificial neural network (ANN) approach.
S40. System according to any of the preceding system embodiments wherein the model is based on a pharmacometrics approach.
S41. System according to any of the preceding system embodiments wherein the model is based on a supervised learning approach.
S42. System according to any of the preceding system embodiments wherein the model is based on a deep learning and/or multi-layer neural network approach.
S43. System according to any of the preceding system embodiments wherein the model is based on an explainable AI concept (XAI).
S44. System according to any of the preceding system embodiment further comprising a trained model that is configured to predict the health status of a young child on the basis of the sensor data.
S45. System according to any of the preceding system embodiments wherein the sensors (1-5) are configured to transmit the signals and/or the sensor data wireless and/or hard-wired.
S46. System according to any of the preceding system embodiments wherein the sensors (1-5) comprise at least one of:
   a non-contact sensor; and/or
   a contact sensor.
S47. System according to any of the preceding system embodiments wherein the sensors (1-5) comprise a camera for capturing frames and/or videos.
S48. System according to any of the preceding system embodiments wherein the sensors (1-5) comprise a camera that is configured to detect physiological and/or skin properties and/or movement of a young child and/or its mother.

System according to any of the preceding system embodiments wherein the sensors (1-5) comprise at least one of:
an amniotic fluid sensor and/or analyzer;
a mother milk sensor and/or analyzer.
S49. System according to any of the preceding system embodiments wherein the sensors (1-5) comprise at least one of:
   laboratory sensors;
   marker sensors.
S50. System according to any of the preceding system embodiments wherein the sensors (1-5) comprise at least one of:
   an infrared sensor;
   an optical monitor, e.g. near infrared spectroscopy
   a photometric sensor;
   a respiratory sensor;
   a breathing analyzer;
   a capnography sensor;
   a cardiac sensor;
   a blood pressure sensor;
   a heart rate sensor
   a hemodynamic sensor;
   an eye tracking sensor
   a movement sensor;
   a temperature sensor;
   a blood oxygen sensor;
   a pulse oximetric sensor;
   a neurological sensor;
   an EEG sensor;
   an electrolyte sensor;
   a hydrolyte sensor;
   a peptide sensor;
   a urine analyzer;
   a stool analyzer;
   a blood gas analyzer;
   a bladder pressure sensor;
   an intracranial pressure sensor;
   a transcutaneous sensor;
   a tissue oxidization sensor and/or
   an ultrasonic and other imaging sensors.
S51. System according to any of the preceding system embodiments wherein the sensors (1-5) or a part thereof are arranged at a point of care (POC) location.
S52. System according to any of the preceding system embodiments wherein the system comprises a sensor data storage (11) that is configured to store the sensor signals and/or sensor data.
S53. System according to the preceding system embodiment wherein the sensor data storage (11) is located local with respect to the sensors (1-5).
S54. System according to any of the preceding system embodiments wherein the system comprises a sensor data processing component (10) that is configured to process the sensor signals into sensor data.
S55. System according to the preceding system embodiment where the data processing component (10) is configured to initiate storage of the sensor data in the sensor data storage (11).
S56. System according to any of the five preceding system embodiments wherein the system comprises a computing component (20) that is configured to communicate with the processing component (10).
S57. System according to any of the six preceding system embodiments wherein the system comprises a computing component (20) that is configured to compute the sensor data of a plurality of processing components (10).
S58. System according to any of the seven preceding system embodiments wherein the system comprises a computing component (20) that is configured to compute the sensor data of a plurality of processing components (10).
S59. System according to any of the preceding system embodiments wherein the system comprises a computing component (20) that is configured to train a software component on the basis of the sensor data of a plurality of processing components (10).
S60. System according to any of the preceding system embodiments wherein the system comprises a computing component (20) that is configured to train a software component on the basis of the sensor data of a plurality of processing components (10) wherein the computing component (20) is located remotely, such as in the cloud.
S61. System according to any of the preceding system embodiments wherein the system comprises a remote storage (21) that is located at least remotely with respect to one set of sensors (1-5).
S62. System according to any of the preceding system embodiments wherein the system comprises a triggering component (22) that is configured to deliver triggering data on the basis of the model.
S63. System according to any of the preceding system embodiments wherein the system comprises a triggering component (22) that is configured to trigger an intervention.
S64. System according to any one of the preceding system embodiments wherein the system is configured to deliver the intervention locally with respect to the sensors (1-5).

Below, method embodiments will be discussed. These embodiments are abbreviated by the letter “M” followed by a number. Whenever reference is herein made to “method embodiments”, these embodiments are meant.
M1. Method for monitoring young children comprising:
   providing signals and/or converted sensor data from a plurality of sensors (1-5);
   managing a health status by a management component (10-26) configured to receive the sensor data on the basis of a model.
M2. Method according to the preceding method embodiment configured for monitoring young children between their conception and the subsequent 1,000 days.
M3. Method according to any of the preceding method embodiments wherein the method is a non-diagnostic method.
M4. Method according to any of the preceding method embodiments wherein the method is a non-diagnostic method.
M5. Method according to any of the preceding method embodiments wherein the method can be performed without the presence of any patient but for testing purposes.
M6. Method according to any of the preceding method embodiments wherein the management component (10-26) comprises a prediction component for predicting the health status on the basis of a model.
M7. Method according to any of the preceding method embodiments wherein the management component (10-26) is further configured to compute at least one of the following data:
   health data from a health data base (21);
   individual data from a storage (22); and/or
   genetic data from a genetic data base (23).
M8. Method according to any of the preceding method embodiments wherein the plurality of sensors (1-5) is configured to be attached to a young child and/or its mother.
M9. Method according to any of the preceding method embodiments wherein the computing component (20) is configured to receive sensor data.
M10. Method according to any of the preceding method embodiments wherein the management component (10-26) further comprises a sensor data storage (11) that is configured for storing the sensor data from the sensors (1-5).
M11. Method according to any of the preceding method embodiments wherein the management component (10-26) further comprises a processing component (10) that is configured for processing sensor data received from the sensors (1-5).
M12. Method according to any of the preceding method embodiments wherein the management component (10-26) further comprises a sensor data storage (11) that is configured for storing the sensor data from the sensors (1-5).
M13. Method according to any of the preceding method embodiments wherein the management component (10-26) further comprises a computing component (20) that is configured for computing the sensor data and for applying the model.
M14. Method according to any of the preceding method embodiments wherein the management component (10-26) further comprises a computing component (20) that is configured for receiving and computing the sensor data and for applying the model.
M15. Method according to any of the preceding method embodiments wherein the management component (10-26) further comprises a computing component (20) that is configured for computing the sensor data and for establishing the model.
M16. Method according to any of the preceding method embodiments wherein the management component (10-26) further comprises a computing component (20) that is configured for receiving and computing the sensor data and for establishing the model.
M17. Method according to any of the preceding method embodiments wherein the management component (10-26) further comprises a computing component (20) that is configured for computing the sensor data and for adjusting the model.
M18. Method according to any of the preceding method embodiments wherein the management component (10-26) further comprises a computing component (20) that is configured for receiving and computing the sensor data and for adjusting the model.
M19. Method according to any of the preceding method embodiments wherein the management component (10-26) further comprises a deciding component (20) that is configured for deciding based on the result of the computing component (20).
M20. Method according to any of the preceding method embodiments wherein the management component (10-26) further comprises a deciding component (20) that is configured for initiating the managing component (10-26) for managing the health status with a different complexity level.
M21. Method according to any of the preceding method embodiments wherein the management component (10-26) further comprises a deciding component (20) that is configured for initiating the managing component (10-26) for managing the health status with a different frequency.
M22. Method according to any of the preceding method embodiments wherein the management component (10-26) further comprises a deciding component (20) that is configured for initiating the managing component (10-26) for triggering at least one medical device.
M23. Method according to any of the preceding method embodiments wherein the management component (10-26) further comprises a deciding component (20) that is configured for initiating the managing component (10-26) for controlling at least one medical device.
M24. Method according to any of the preceding method embodiments wherein the computing component (20) is located locally and/or remotely.
M25. Method according to any of the preceding method embodiments wherein the management component (10-26) is located locally and/or remotely.
M26. Method according to any of the preceding method embodiments wherein the management component (10-26) is configured for providing a diagnosis.
M27. Method according to any of the preceding method embodiments wherein the management component (10-26) is configured for providing a therapy.
M28. Method according to any of the preceding method embodiments wherein the management component (10-26) is configured for logging the health status.
M29. Method according to any of the preceding method embodiments wherein the management component (10-26) is configured for predicting the health status.
M30. Method according to any of the preceding method embodiments wherein the management component (10-26) is configured for predicting the health status and for training the model on the basis of the previously predicted and the actual health status.
M31. Method according to any of the preceding method embodiments wherein the management component (10-26) is further configured to trigger an intervention on the basis of the prediction.
M32. Method according to the preceding method embodiment wherein the intervention comprises an alarm signal.
M33. Method according to the two preceding method embodiments wherein the intervention comprises an acoustical signal.
M34. Method according to the three preceding method embodiments wherein the intervention comprises a treatment proposal, preferably configured to be displayed on a screen (22).
M35. Method according to the four preceding method embodiments wherein the intervention comprises a control signal to a respective treatment component (22).
M36. Method according to any of the preceding method embodiments further comprising a model that is configured to predict the health status of a young child on the basis of the sensor data.
M37. Method according to any of the preceding method embodiments wherein the management component (10-26) is configured to train the model.
M38. Method according to any of the preceding method embodiments wherein the management component (10-26) is configured to train the model on the basis of the sensor data.
M39. Method according to any of the preceding method embodiments wherein the management component (10-26) is configured to train the model on the basis of the sensor data and other variables.
M40. Method according to any of the preceding method embodiments wherein the model is based on a Bayesian statistical approach.
M41. Method according to any of the preceding method embodiments wherein the model is based on an artificial neural network (ANN) approach.
M42. Method according to any of the preceding method embodiments wherein the model is based on a pharmacometrics approach.
M43. Method according to any of the preceding method embodiments wherein the model is based on a supervised learning approach.
M44. Method according to any of the preceding method embodiments wherein the model is based on a deep learning and/or multi-layer neural network approach.
M45. Method according to any of the preceding method embodiments wherein the model is based on an explainable AI concept (XAI).
M46. Method according to any of the preceding method embodiment further comprising a trained model that is configured to predict the health status of a young child on the basis of the sensor data.
M47. Method according to any of the preceding method embodiments wherein the sensors (1-5) are configured to transmit the signals and/or the sensor data wireless and/or hard-wired.
M48. Method according to any of the preceding method embodiments wherein the sensors (1-5) comprise at least one of:
   a non-contact sensor; and/or
   a contact sensor.
M49. Method according to any of the preceding method embodiments wherein the sensors (1-5) comprise at least one of: a camera for capturing frames and/or videos.
M50. Method according to any of the preceding method embodiments wherein the sensors (1-5) comprise at least one of: a camera that is configured to detect physiological and/or skin properties and/or movement of a young child and/or its mother.
M51. Method according to any of the preceding method embodiments wherein the sensors (1-5) comprise at least one of:
   an amniotic fluid sensor and/or analyzer;
   a mother milk sensor and/or analyzer.
M52. Method according to any of the preceding method embodiments wherein the sensors (1-5) comprise at least one of:
   laboratory sensors;
   marker sensors.
M53. Method according to any of the preceding method embodiments wherein the sensors (1-5) comprise at least one of:
   an infrared sensor;
   a photometric sensor;
   a respiratory sensor;
   a breathing analyzer;
   a capnography sensor;
   a cardiac sensor;
   a blood pressure sensor;
   a hemodynamic sensor;
   a movement sensor;
   a temperature sensor;
   a blood oxygen sensor;
   a pulse oximetric sensor;
   a neurological sensor;
   an EEG sensor;
   an electrolyte sensor;
   a hydrolyte sensor;
   a peptide sensor;
   a urine analyzer;
   a stool analyzer;
   a blood gas analyzer;
   a bladder pressure sensor;
   an intracranial pressure sensor;
   a transcutaneous sensor;
   a tissue oxidization sensor and/or an ultrasonic and other imaging sensors.
M54. Method according to any of the preceding method embodiments wherein the sensors (1-5) or a part thereof are arranged at a point of care (POC) location.
M55. Method according to any of the preceding method embodiments wherein the method comprises a sensor data storage (11) that is configured to store the sensor signals and/or sensor data.
M56. Method according to the preceding method embodiment wherein the sensor data storage (11) is located local with respect to the sensors (1-5).
M57. Method according to any of the preceding method embodiments wherein the method comprises a sensor data processing component (10) that is configured to process the sensor signals into sensor data.
M58. Method according to the preceding method embodiment where the data processing component (10) is configured to initiate storage of the sensor data in the sensor data storage (11).
M59. Method according to any of the five preceding method embodiments wherein the system comprises a computing component (20) that is configured to communicate with the processing component (10).
M60. Method according to any of the six preceding method embodiments wherein the system comprises a computing component (20) that is configured to compute the sensor data of a plurality of processing components (10).
M61. Method according to any of the seven preceding method embodiments wherein the system comprises a computing component (20) that is configured to compute the sensor data of a plurality of processing components (10).
M62. Method according to any of the preceding method embodiments wherein the system comprises a computing component (20) that is configured to train a software component on the basis of the sensor data of a plurality of processing components (10).
M63. Method according to any of the preceding method embodiments wherein the system comprises a computing component (20) that is configured to train a software component on the basis of the sensor data of a plurality of processing components (10) wherein the computing component (20) is located remotely, such as in the cloud.
M64. Method according to any of the preceding method embodiments wherein the system comprises a remote storage (21) that is located at least remotely with respect to one set of sensors (1-5).
M65. Method according to any of the preceding method embodiments wherein the system comprises a triggering component (22) that is configured to deliver triggering data on the basis of the model.
M66. Method according to any of the preceding method embodiments wherein the system comprises a triggering component (22) that is configured to trigger an intervention.
M67. Method according to any one of the preceding method embodiments wherein the system is configured to deliver the intervention locally with respect to the sensors (1-5).

Below, use embodiments will be discussed. These embodiments are abbreviated by the letter “U” followed by a number. Whenever reference is herein made to “use embodiments”, these embodiments are meant.
U1. Use of the system according to any of the preceding system embodiments for diagnosing young children by applying the system according to any of the preceding system embodiments.
U2. Use of the method according to any of the preceding method embodiments for diagnosing young children by applying the method according to any of the preceding method embodiments.
U3. Use of the system according to any of the preceding system embodiments for a therapy of young children by applying the system according to any of the preceding system embodiments.
U4. Use of the method according to any of the preceding method embodiments for a therapy of young children by applying the method according to any of the preceding method embodiments.

Below, computer related product embodiments will be discussed. These embodiments are abbreviated by the letter “C” followed by a number. Whenever reference is herein made to “computer related product embodiments”, these embodiments are meant.
C1. A computer related product with a program that is configured for carrying out the method according to any of the preceding method embodiments.
C2. A computer related product with a program and a model that is configured for carrying out the method according to any of the preceding method embodiments where the model is based on an explicable artificial intelligence concept (XAI).

Below, diagnostic and monitoring embodiments will be discussed. These embodiments are abbreviated by the letter “D” followed by a number. Whenever reference is herein made to “diagnostic embodiments”, these embodiments are meant.
D1. Method of a diagnostic treatment of a patient with a pharmaceutical component that has been provided by a method according to any of the preceding method embodiments.
D2. Method of a diagnostic treatment of a patient with a pharmaceutical component that has been provided by a system according to any of the preceding system embodiments.
D3. Method of a diagnostic treatment of a patient with a software that has been provided by a computer related product according to the preceding computer related product embodiment.

Below, therapeutic embodiments will be discussed. These embodiments are abbreviated by the letter “T” followed by a number. Whenever reference is herein made to “therapeutic embodiments”, these embodiments are meant.
T1. Method of a therapeutic treatment of a patient with a pharmaceutical component that has been provided by a method according to any of the preceding method embodiments.
T2. Method of a therapeutic treatment of a patient with a pharmaceutical component that has been provided by a system according to any of the preceding system embodiments.
T3. Method of a therapeutic treatment of a patient with a software that has been provided by a computer related product according to the preceding computer related product embodiment.

The present invention will now be described with reference to the accompanying drawings, which illustrate embodiments of the invention. These embodiments should only exemplify, but not limit, the present invention.

### FIGURE DESCRIPTION

FIG. 1 schematically exemplifies a flowchart in accordance with an embodiment according to the invention.

FIG. 2 shows another principal sketch in accordance with an embodiment according to the invention.

FIG. 3 depicts another principal embodiment in accordance with the invention.

FIG. 4 exemplifies a further principal embodiment in accordance with the invention.

### DESCRIPTION OF PREFERRED EMBODIMENTS AS EXEMPLIFIED IN THE FIGURES

It is noted that not all the drawings carry all the reference signs. Instead, in some of the drawings, some of the reference signs have been omitted for sake of brevity and simplicity of illustration. Embodiments of the present invention will now be described with reference to the accompanying drawings.

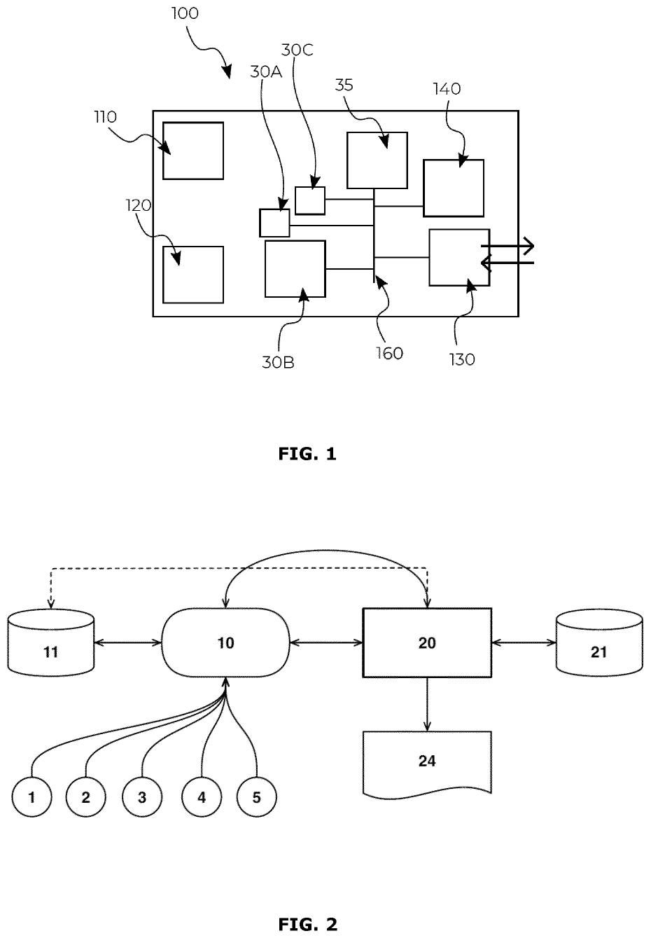

FIG. 1 provides a schematic of a computing device 100. The computing device 100 may comprise a computing unit 35, a first data storage unit 30A, a second data storage unit 30B and a third data storage unit 30C.

The computing device 100 can be a single computing device or an assembly of computing devices. The computing device 100 can be locally arranged or remotely, such as a cloud solution.

On the different data storage units 30 the different data can be stored. Additional data storages can be also provided and/or the ones mentioned before can be combined at least in part.

The computing unit 35 can access the first data storage unit 30A, the second data storage unit 30B and the third data storage unit 30C through the internal communication channel 160, which can comprise a bus connection 160.

The computing unit 30 may be single processor or a plurality of processors, and may be, but not limited to, a CPU (central processing unit), GPU (graphical processing unit), DSP (digital signal processor), APU (accelerator processing unit), ASIC (application-specific integrated circuit), ASIP (application-specific instruction-set processor) or FPGA (field programable gate array). The first data storage unit 30A may be singular or plural, and may be, but not limited to, a volatile or non-volatile memory, such as a random access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The second data storage unit 30B may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

The third data storage unit 30C may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM).

It should be understood that generally, the first data storage unit 30A (also referred to as encryption key storage unit 30A), the second data storage unit 30B (also referred to as data share storage unit 30B), and the third data storage unit 30C (also referred to as decryption key storage unit 30C) can also be part of the same memory. That is, only one general data storage unit 30 per device may be provided, which may be configured to store the respective encryption key (such that the section of the data storage unit 30 storing the encryption key may be the encryption key storage unit 30A), the respective data element share (such that the section of the data storage unit 30 storing the data element share may be the data share storage unit 30B), and the respective decryption key (such that the section of the data storage unit 30 storing the decryption key may be the decryption key storage unit 30A).

In some embodiments, the third data storage unit 30C can be a secure memory device 30C, such as, a self-encrypted memory, hardware-based full disk encryption memory and the like which can automatically encrypt all of the stored data. The data can be decrypted from the memory component only upon successful authentication of the party requiring to access the third data storage unit 30C, wherein the party can be a user, computing device, processing unit and the like. In some embodiments, the third data storage unit 30C can only be connected to the computing unit 35 and the computing unit 35 can be configured to never output the data received from the third data storage unit 30C. This can ensure a secure storing and handling of the encryption key (i.e. private key) stored in the third data storage unit 30C.

In some embodiments, the second data storage unit 30B may not be provided but instead the computing device 100 can be configured to receive a corresponding encrypted share from the database 60. In some embodiments, the computing device 100 may comprise the second data storage unit 30B and can be configured to receive a corresponding encrypted share from the database 60.

The computing device 100 may comprise a further memory component 140 which may be singular or plural, and may be, but not limited to, a volatile or non-volatile memory, such as a random-access memory (RAM), Dynamic RAM (DRAM), Synchronous Dynamic RAM (SDRAM), static RAM (SRAM), Flash Memory, Magneto-resistive RAM (MRAM), Ferroelectric RAM (F-RAM), or Parameter RAM (P-RAM). The memory component 140 may also be connected with the other components of the computing device 100 (such as the computing component 35) through the internal communication channel 160.

Further the computing device 100 may comprise an external communication component 130. The external communication component 130 can be configured to facilitate sending and/or receiving data to/from an external device (e.g. backup device, recovery device, database). The external communication component 130 may comprise an antenna (e.g. WIFI antenna, NFC antenna, 2G/3G/4G/5G antenna and the like), USB port/plug, LAN port/plug, contact pads offering electrical connectivity and the like. The external communication component 130 can send and/or receive data based on a communication protocol which can comprise instructions for sending and/or receiving data. Said instructions can be stored in the memory component 140 and can be executed by the computing unit 35 and/or external communication component 130. The external communication component 130 can be connected to the internal communication component 160. Thus, data received by the external communication component 130 can be provided to the memory component 140, computing unit 35, first data storage unit 30A and/or second data storage unit 30B and/or third data storage unit 30C. Similarly, data stored on the memory component 140, first data storage unit 30A and/or second data storage unit 30B and/or third data storage unit 30C and/or data generated by the computing unit 35 can be provided to the external communication component 130 for being transmitted to an external device.

In addition, the computing device 100 may comprise an input user interface 110 which can allow the user of the computing device 100 to provide at least one input (e.g. instruction) to the computing device 100. For example, the input user interface 110 may comprise a button, keyboard, trackpad, mouse, touchscreen, joystick and the like.

Additionally, still, the computing device 100 may comprise an output user interface 120 which can allow the computing device 100 to provide indications to the user. For example, the output user interface 110 may be a LED, a display, a speaker and the like.

The output and the input user interface 100 may also be connected through the internal communication component 160 with the internal component of the device 100.

The processor may be singular or plural, and may be, but not limited to, a CPU, GPU, DSP, APU, or FPGA. The memory may be singular or plural, and may be, but not limited to, being volatile or non-volatile, such an SDRAM, DRAM, SRAM, Flash Memory, MRAM, F-RAM, or P-RAM.

The data processing device can comprise means of data processing, such as, processor units, hardware accelerators and/or microcontrollers. The data processing device 20 can comprise memory components, such as, main memory (e.g. RAM), cache memory (e.g. SRAM) and/or secondary memory (e.g. HDD, SDD). The data processing device can comprise busses configured to facilitate data exchange between components of the data processing device, such as, the communication between the memory components and the processing components. The data processing device can comprise network interface cards that can be configured to connect the data processing device to a network, such as, to the Internet. The data processing device can comprise user interfaces, such as:
output user interface, such as:
   screens or monitors configured to display visual data (e.g. displaying graphical user interfaces of the questionnaire to the user),
   speakers configured to communicate audio data (e.g. playing audio data to the user),
input user interface, such as:
   camera configured to capture visual data (e.g. capturing images and/or videos of the user),
   microphone configured to capture audio data (e.g. recording audio from the user),
   keyboard configured to allow the insertion of text and/or other keyboard commands (e.g. allowing the user to enter text data and/or other keyboard commands by having the user type on the keyboard) and/or trackpad, mouse, touchscreen, joystick-configured to facilitate the navigation through different graphical user interfaces of the questionnaire.

The data processing device can be a processing unit configured to carry out instructions of a program. The data processing device can be a system-on-chip comprising processing units, memory components and busses. The data processing device can be a personal computer, a laptop, a pocket computer, a smartphone, a tablet computer. The data processing device can be a server, either local and/or remote. The data processing device can be a processing unit or a system-on-chip that can be interfaced with a personal computer, a laptop, a pocket computer, a smartphone, a tablet computer and/or user interface (such as the upper-mentioned user interfaces).

FIG. 2 exemplifies a principal setup of several components in line with an embodiment according to the present invention. As already mentioned, this is an example only but is intended to show a principal working example.

It is an aspect of the invention to have at least two or even more sensors 1-5 arranged to be able to monitor a young child and/or its mother (not shown). The newborn is also not necessary in order to operate the system or to run the method as specified before and below.

The sensors 1-5 can be of different nature and can be configured to determine, analyze, measure etc. either directly, indirectly or in combination different variables, variants, covariates of a young child and/or its mother. The sensors 1-5 deliver signals that are either converted by the sensor and/or a downstream component into sensor data over time.

The downstream component can be a processing component 10 that is configured to communicate with the sensors hardwired and/or wireless. The processing component 10 can initiate and organize storage of the sensor data in a sensor data storage 11. This sensor data storage 11 can be arranged locally with respect to the sensors 1-5. However, it can also be arranged remotely, such as in the cloud.

There can be an additional computing component 20 that can be arranged remotely with respect to the sensors 1-5 and/or with respect to the processing component 10. An advantage can be that the sensor data is collected and stored in different locations and the computing component 20 can then obtain (push and/or pull) the aggregated sensor data from each sensor data storage 11 when needed. This can be done in case the computing component 20 is processing and/or training a model that is needed.

The dotted line in FIG. 1 stands for an optional direct access of the sensor data storage 11 by the computing component 20. This can be unilateral or bilateral. In the latter case the computing component 20 can modify or delete sensor data in the sensor data storage 11.

For the training and/or other purposes, there is arranged another remote storage 21 that can store the software underlying the model and/or any sensor data and/or any other data.

In the example shown, the computing component 20 is not only training the software but also analyzing the actual sensor data from the sensors 1-5. It is within the meaning of the invention to also convey the trained software to the locally arranged processing component 10 that is then analyzing the sensor data locally and/or any other processing component (not shown).

In the example shown, the computing component is delivering information, signals, data and/or any other content in order to deliver the result of the analyzing. In the example shown, the data is delivered to a triggering component or information component 24.

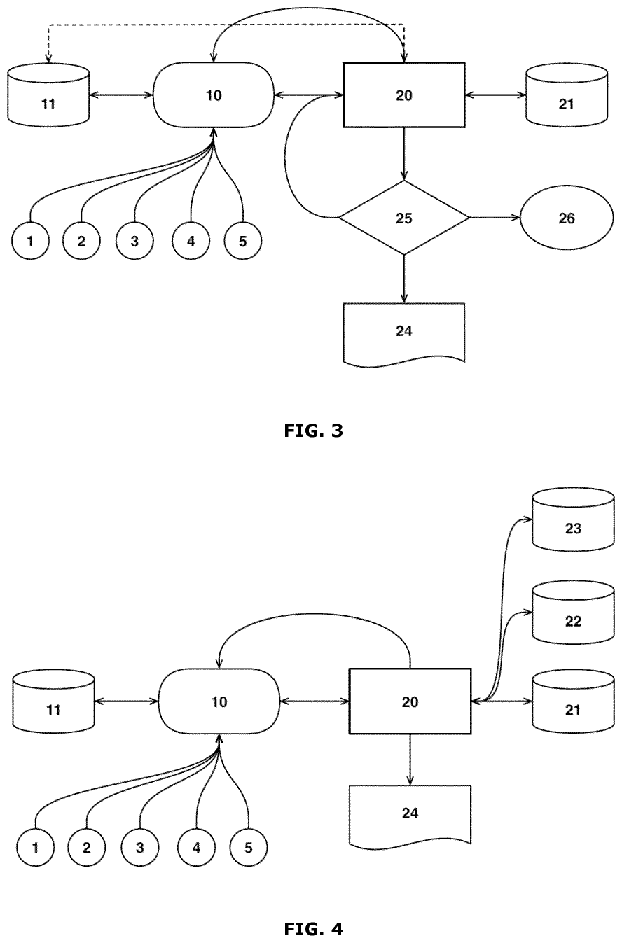

FIG. 3 shows another embodiment in line with the present invention. The major difference that may also be implemented in other embodiments shown or described is a deciding component 25 that is configured to decide further controls or steps depending on the result received by the computing component 20.

In one case, the deciding component can receive information from the computing component 20 that makes it necessary or appropriate to trigger the computing component 20 to compute a more complex, a less complex model and/or to compute the sensor data more frequent or less frequent. This is illustrated by the arrow leaving the left-hand side of the deciding component 25.

On the opposite side an arrow goes to a trigger component 26 that is configured to trigger other medical devices for a further treatment of the young child and/or its mother.

The arrow to the bottom exemplifies the further procedure as discussed with respect to FIG. 1.

FIG. 4 shows further databases 21-23 that may be consulted by the system and method according to the present invention. This is just an example. Instead, one database or the cloud can deliver the same information. The databases 21-23 can comprise different data, such as genomic or genetic data, epigenetic data, known health data, health data for an actual young child and/or its mother that can be actualized intermittently or constantly or both.

In the example shown, there can be provided health data from a health data base 21; individual data from a storage 22 and/or genetic data from a genetic data base 23. The data can be also combined in fewer data bases or split up in more data bases. The individual data means data from the young child and/or its mother. Genetic data can comprise any genetic, genomic, epigenetic data etc. already known.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be accidental. That is, unless otherwise specified or unless clear to the skilled person, the order in which steps are recited may be accidental. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

1. Method for monitoring young children comprising:
providing signals and/or converted sensor data from a plurality of sensors (1-5);
managing a health status by a management component (10-26) configured to receive the sensor data on the basis of a model.

2. Method according to claim 1 wherein the method comprises monitoring young children between their conception and the subsequent 1,000 days.

3. Method according to claim 1 wherein the management component (10-26) comprises a prediction component for predicting the health status on the basis of a model.

4. Method according to claim 1 or claim 3 wherein the management component (10-26) is further configured to compute at least one of the following data:
health data from a health data base (21);
individual data from a storage (22); and/or
genetic data from a genetic data base (23).

5. Method according to claim 1 wherein the plurality of sensors (1-5) is configured to be attached to a young child and/or its mother.

6. Method according to claim 1 wherein the method comprises receiving sensor data by a computing component (20).

7. Method according to claim 1 wherein the management component (10-26) further comprises a sensor data storage (11) that is configured for storing the sensor data from the sensors (1-5).

8. Method according to claim 1 wherein the management component (10-26) further comprises a processing component (10) that is configured for processing sensor data received from the sensors (1-5).

9. Method according to claim 1 wherein the management component (10-26) further comprises a computing component (20) that is configured for computing the sensor data and for applying the model.

10. Method according to claim 1 wherein the management component (10-26) further comprises a computing component (20) that is configured for computing the sensor data and for establishing the model.

11. Method according to claim 1 wherein the management component (10-26) further comprises a computing component (20) that is configured for computing the sensor data and for adjusting the model.

12. Method according to claim 1 wherein the management component (10-26) further comprises a deciding component (20) that is configured for initiating the managing component (10-26) for managing the health status with a different complexity level.

13. Method according to claim 1 wherein the management component (10-26) further comprises a deciding component (20) that is configured for initiating the managing component (10-26) for managing the health status with a different frequency.

14. System for monitoring young children comprising:
a plurality of sensors (1-5) providing signals that are configured to be converted into sensor data;
a management component (10-26) configured for receiving the sensor data and for managing a health status and or disease progression on the basis of a model.

15. System according to claim 14 wherein the management component (10-26) comprises a prediction component for predicting the health status on the basis of a model.

16. System according to claim 14 wherein the management component (10-26) is further configured to compute at least one of the following data:
health data from a health data base (21);
individual data from a storage (22); and/or
genetic data from a genetic data base (23).

17. System according to claim 14 wherein the plurality of sensors (1-5) is configured to be attached to a young child and/or its mother.

18. System according to claim 14 wherein the management component (10-26) further comprises a processing component (10) that is configured for processing sensor data received from the sensors (1-5).

19. System according to claim 14 wherein the management component (10-26) further comprises a sensor data storage (11) that is configured for storing the sensor data from the sensors (1-5).

20. System according to claim 14 wherein the management component (10-26) further comprises a computing component (20) that is configured for computing the sensor data and for applying the model.

21. System according to claim 14 wherein the management component (10-26) further comprises a computing component (20) that is configured for computing the sensor data and for establishing the model.

22. System according to claim 14 wherein the management component (10-26) further comprises a computing component (20) that is configured for computing the sensor data and for adjusting the model.

23. System according to claim 14 wherein the management component (10-26) further comprises a deciding component (20) that is configured for initiating the managing component (10-26) for managing the health status with a different complexity level.

24. System according to claim 14 wherein the management component (10-26) further comprises a deciding component (20) that is configured for initiating the managing component (10-26) for managing the health status with a different frequency.

25. A computer related product for carrying out the method of claim 1.

26. A computer related product for carrying of claim 1 wherein the model is based on an explicable AI concept (AIX).
